(19) United States
(12) Patent Application Publication
(10) Pub. No.: US 2025/0083757 A1
(43) Pub. Date: Mar. 13, 2025

(54) RETRACTABLE WHEELS FOR ROBOTIC SURGICAL SYSTEM

(71) Applicant: ForSight Robotics Ltd., Yokneam Illit (IL)

(72) Inventors: Dima RADCHENKO, Haifa (IL); Andrey DUMCHIN, Ramat Yishai (IL); Ofer ARNOLD, Ma'ale Zvia (IL)

(21) Appl. No.: 18/825,382

(22) Filed: Sep. 5, 2024

Related U.S. Application Data
(60) Provisional application No. 63/537,053, filed on Sep. 7, 2023.

(51) Int. Cl. B62D 61/12; A61B 34/30

(52) U.S. Cl. CPC B62D 61/12; A61B 34/30

(57) ABSTRACT

An apparatus includes a chassis, one or more robotic arms mounted to the chassis and configured to perform robotic surgery, multiple wheel units comprising respective wheels and configured to alternate the wheels between a lower position, in which the wheels support the chassis, and an upper position, and one or more legs connected to the chassis and configured to support the chassis while the wheels are in the upper position. Other embodiments are also described.

### CROSS-REFERENCE TO RELATED APPLICATIONS

The present application claims the benefit of U.S. Provisional Application 63/537,053, entitled “Retractable wheels for robotic surgical system,” filed Sep. 7, 2023, whose disclosure is incorporated herein by reference.

### FIELD OF THE INVENTION

The present invention is related generally to the field of robotic surgery, and particularly to the design of robotic surgical systems.

### BACKGROUND OF THE INVENTION

In some types of robotic surgery, one or more robotic arms manipulate surgical tools under the control of an operator.

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and the face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more (e.g., 2-3) incisions, typically including at least one larger incision having a three-planar form, are made in the cornea of the eye. The incisions are typically made using a specialized blade, which is called a keratome blade. Subsequently, another anesthetic, such as lidocaine, is injected into the anterior chamber of the eye via the corneal incisions. Following this step, the pupil is dilated, and a viscoelastic injection is applied via the corneal incisions. The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed, using one or more tools inserted via the corneal incisions. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incisions, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. When the phacoemulsification is complete, the remaining lens cortex (i.e., the outer layer of the lens) and viscoelastic material is aspirated from the capsule. During the phacoemulsification and the aspiration, aspirated fluids are typically replaced with irrigation of a balanced salt solution, in order to maintain fluid pressure in the anterior chamber.

In some cases, if deemed to be necessary, the capsule is polished. Subsequently, the intraocular lens (IOL) is inserted into the capsule. The IOL is typically foldable and is inserted in a folded configuration, before unfolding inside the capsule. If necessary, one or more of the incisions are sealed by elevating the pressure inside the bulbus oculi (i.e., the globe of the eye), causing the internal tissue to be pressed against the external tissue of the incisions, such as to force closed the incisions.

### SUMMARY OF THE INVENTION

Some embodiments of the present invention include a robotic surgical apparatus, which includes a chassis and one or more robotic units supported by the chassis. Typically, it is necessary to equip the chassis with wheels, such that the chassis may be moved to and from the operating table. However, when supported by the wheels, the chassis may not be stable enough to allow the robotic units to perform a surgical procedure safely.

One hypothetical solution is to provide the chassis with retractable legs, which can support the chassis during the surgical procedure. However, by virtue of their retractability, the legs may not be stable enough to stabilize the chassis in the event that side forces and/or other forces are applied to the chassis.

To address this challenge, some embodiments of the present invention provide an apparatus including a chassis, one or more robotic arms mounted to the chassis and configured to perform robotic surgery, multiple wheel units including respective wheels and configured to alternate the wheels between a lower position, in which the wheels support the chassis, and an upper position, and multiple legs connected to the chassis and configured to support the chassis while the wheels are in the upper position. In some embodiments, each of the wheel units further includes a rotating element coupled to the wheel of the wheel unit from above and configured to rotate between a wheel-down orientation, in which the wheel is in the lower position, and a wheel-up orientation, in which the wheel is in the upper position.

There is therefore provided, in accordance with some embodiments of the present invention, an apparatus, including a chassis, one or more robotic arms mounted to the chassis and configured to perform robotic surgery, multiple wheel units including respective wheels and configured to alternate the wheels between a lower position, in which the wheels support the chassis, and an upper position, and one or more legs connected to the chassis and configured to support the chassis while the wheels are in the upper position.

In some embodiments, a maximum height of each of the wheel units from a base of the chassis is less than 15 cm.

In some embodiments, each of the wheel units further includes a rotating element coupled to the wheel of the wheel unit from above and configured to rotate between a wheel-down orientation, in which the wheel is in the lower position, and a wheel-up orientation, in which the wheel is in the upper position.

In some embodiments, each of the wheel units further includes a wheel-down sensor configured to output a wheel-down signal indicating whether the rotating element is in the wheel-down orientation.

In some embodiments, each of the wheel units further includes a wheel-up sensor configured to output a wheel-up signal indicating whether the rotating element is in the wheel-up orientation.

In some embodiments, each of the wheel units further includes a pivot coupled to the chassis, the rotating element includes a first portion, which is coupled to the wheel, and a second portion, and the rotating element is coupled to the pivot between the first portion and the second portion, such that the rotating element is configured to rotate about the pivot.

In some embodiments, each of the wheel units further includes:
an axle;
an eccentric element coupled eccentrically to the axle; and
a motor coupled to the chassis and configured to rotate the axle so as to move the eccentric element between a supporting position, in which the eccentric element contacts the second portion of the rotating element so as to support the rotating element in the wheel-down orientation, and a non-supporting position, in which the eccentric element does not inhibit the rotating element from rotating to the wheel-up orientation.

In some embodiments, each of the wheel units further includes a supporting-position sensor configured to output a higher-position signal indicating whether the eccentric element is in the supporting position.

In some embodiments, in the supporting position, the eccentric element leans against the chassis.

In some embodiments, each of the wheel units further includes at least one tension spring coupled between the second portion of the rotating element and the chassis and configured to pull down the second portion of the rotating element.

In some embodiments, each of the wheel units further includes a lever coupled to the chassis and configured for rotation, by a user, so as to rotate the rotating element from the wheel-up orientation.

In some embodiments, each of the wheel units further includes a tension spring coupled between the lever and the chassis and configured to pull the lever away from the rotating element.

In some embodiments, each of the wheel units further includes a locking element coupled to the lever and configured to lock against the chassis such that the lever supports the rotating element following the rotation of the lever by the user.

In some embodiments, the chassis includes a protrusion, and the locking element is configured to lock against the chassis by fitting around the protrusion.

In some embodiments, each of the wheel units further includes a release handle coupled to the locking element and configured for rotation, by the user, so as to release the locking element from the chassis.

In some embodiments, each of the wheel units further includes a tension spring coupled between the locking element and the chassis and configured to facilitate the locking of the locking element against the chassis by pulling the locking element.

In some embodiments,
the wheel-down orientation is a first wheel-down orientation, and
the lever is configured for rotation so as to rotate the rotating element from the wheel-up orientation to a second wheel-down orientation in which the wheel supports the chassis,
   the second portion of the rotating element being higher in the first wheel-down orientation than in the second wheel-down orientation.

In some embodiments,
each of the wheel units further includes one or more sensors configured to output respective signals, and
the apparatus further includes a controller, configured to:
   receive the signals from the sensors, and
   based on the signals, identify a current orientation of the rotating element from a group of orientations consisting of: the wheel-up orientation, the first wheel-down orientation, and the second wheel-down orientation.

In some embodiments, the controller is further configured to rotate the rotating element to the first wheel-down orientation in response to ascertaining that the rotating element is in the second wheel-down orientation.

In some embodiments,
each of the wheel units further includes a locking element coupled to the lever and configured to lock against the chassis such that the lever supports the rotating element in the second wheel-down orientation, and
the controller is further configured to output an alert indicating a need to release the locking element, subsequently to rotating the rotating element to the first wheel-down orientation.

There is further provided, in accordance with some embodiments of the present invention, an apparatus including a chassis and multiple wheel units including respective wheels and configured to alternate the wheels between a lower position, in which the wheels support the chassis, and an upper position. Each of the wheel units further includes a rotating element coupled to the wheel of the wheel unit from above and configured to rotate between a wheel-down orientation, in which the wheel is in the lower position, and a wheel-up orientation, in which the wheel is in the upper position. The apparatus further includes one or more legs connected to the chassis and configured to support the chassis while the wheels are in the upper position.

The present invention will be more fully understood from the following detailed description of embodiments thereof, taken together with the drawings, in which:

### BRIEF DESCRIPTION OF THE DRAWINGS

FIG. 1A is a schematic illustration of an apparatus comprising multiple wheel units, in accordance with some embodiments of the present invention;

FIG. 1B is a schematic illustration of a robotic surgical apparatus, in accordance with some embodiments of the present invention;

FIGS. 2A-2B are schematic illustrations of a wheel unit, in accordance with some embodiments of the present invention; and

FIGS. 3A-3B are schematic illustrations of a portion of a wheel unit, in accordance with some embodiments of the present invention.

### DETAILED DESCRIPTION

### Overview

Some embodiments of the present invention include a robotic surgical apparatus, which includes a chassis and one or more robotic units supported by the chassis. Typically, it is necessary to equip the chassis with wheels, such that the chassis may be moved to and from the operating table. However, when supported by the wheels, the chassis may not be stable enough to allow the robotic units to perform a surgical procedure safely.

One hypothetical solution is to provide the chassis with retractable legs, which can support the chassis during the surgical procedure. However, by virtue of their retractability, the legs may not be stable enough to stabilize the chassis in the event that side forces and/or other forces are applied to the chassis.

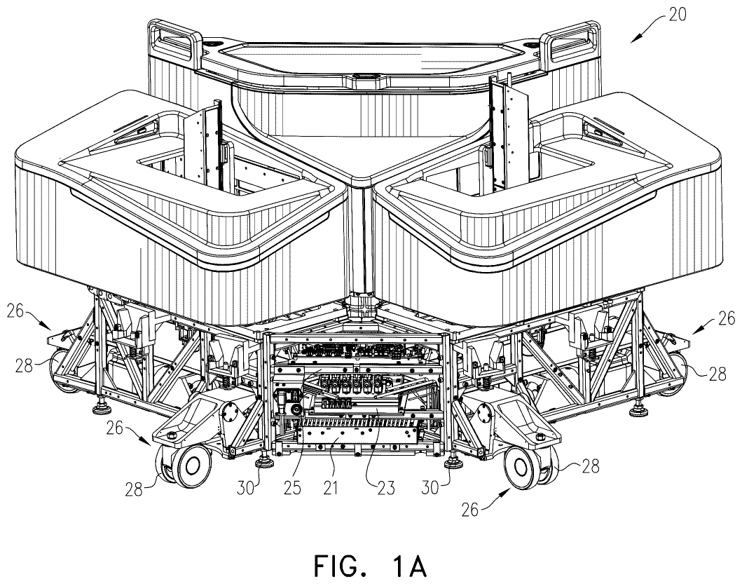

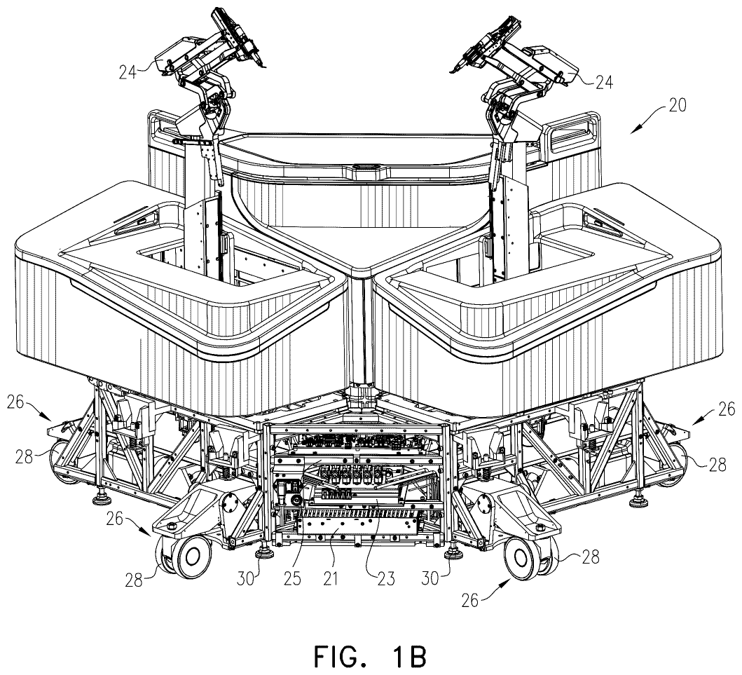

Hence, some embodiments of the present invention provide a robotic surgical apparatus, shown in FIG. 1B with reference number 20, having retractable wheels 28 and stationary legs 30 that do not retract. After wheeling the apparatus into position for the surgical procedure, the wheels are raised such that the apparatus is supported, stably, by the stationary legs. Following the procedure, the wheels are lowered, and the apparatus is then wheeled away from the operating table.

Although the scope of the present invention includes the use of any suitable mechanism for raising and lowering the wheels, certain components of the apparatus may constrain the maximum allowable height of the mechanism. Thus, for example, although the scope of the present invention includes the use of a vertically-aligned linear piston, the use of such a mechanism may be impractical.

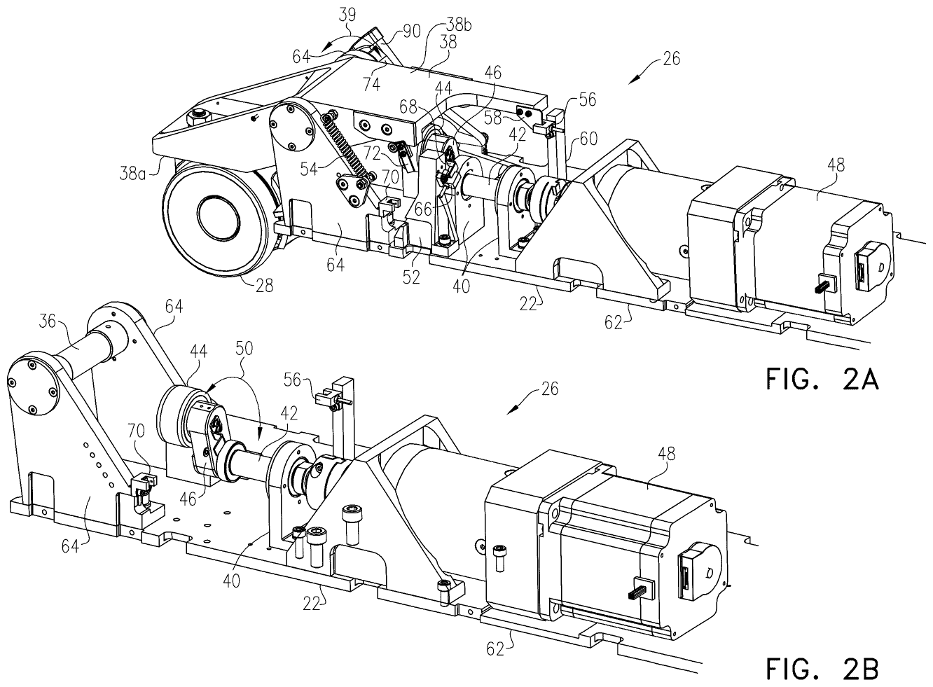

To address this challenge, therefore, some embodiments of the present invention provide, for each of the wheels, a particular wheel-retracting mechanism that occupies relatively little vertical space. The mechanism comprises a rotating element 38 (FIG. 2A) coupled, from above, to the wheel. The rotating element is configured to rotate (e.g., about a fulcrum, shown in FIG. 2B with reference number 36), so as to raise or lower the wheel.

In general, the rotating of the rotating element may be driven in any suitable way. For example, as shown in FIGS. 2A-2B, the mechanism may further comprise a horizontal axle 42, an eccentric element 44 coupled eccentrically to the axle underneath the rotating element at the side of the fulcrum opposite the wheel, and a motor 48. As the motor rotates the axle, the eccentric element (by virtue of its eccentric coupling to the axle) moves arcedly through a vertical plane, either toward or away from the rotating element. Thus, the eccentric element may either (on its way up) push against the rotating element so as to lower the wheel, or (on its way down) release the rotating element such that the rotating element may rotate back to its default orientation in which the wheel is raised.

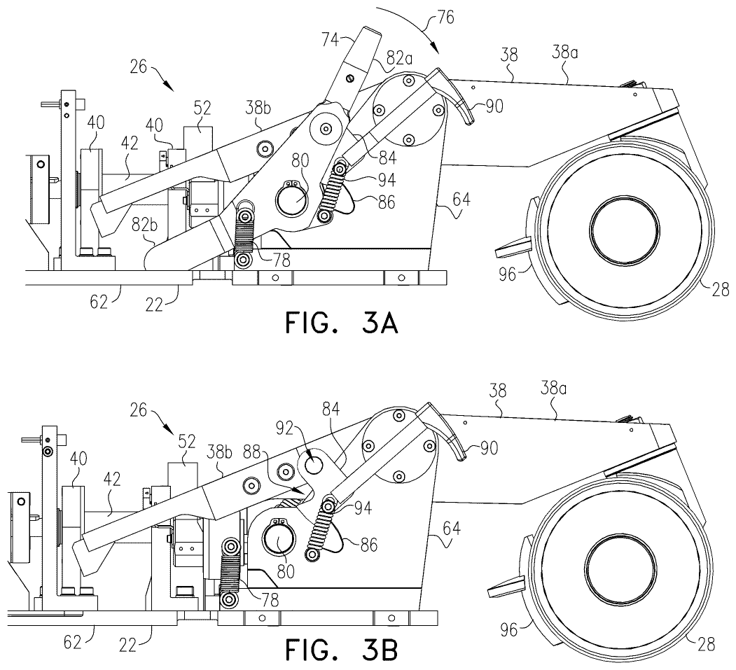

Typically, the apparatus further comprises respective levers 74 (FIG. 3A) for the wheels, which may be manually operated so as to lower the wheels, e.g., in the event of a power failure.

Typically, the apparatus further comprises one or more sensors configured to sense the position and/or orientation of the rotating element, the eccentric element, and/or any other element of the apparatus. Based on signals from the sensors, a controller may discriminate between the three possible states of the apparatus: a first state in which the wheels were lowered automatically (e.g., using the eccentric element), a second state in which the wheels were lowered manually, and a third state in which the wheels are raised (and the apparatus is supported by the legs). Advantageously, ascertaining the state of the apparatus may allow the controller to control the apparatus more effectively.

Alternatively to a surgical robot, the embodiments described briefly above and further described below with reference to the figures may be applied to any other type of wheeled apparatus for which stabilization is required.

### Robotic Surgical Apparatus

Reference is initially made to FIG. 1A, which is a schematic illustration of an apparatus 20 comprising multiple wheel units 26, in accordance with some embodiments of the present invention. Reference is also made to FIG. 1B, which is a schematic illustration of apparatus 20 functioning as a robotic surgical apparatus, in accordance with some embodiments of the present invention.

Apparatus 20 comprises a chassis 22. In some embodiments, as shown in FIG. 1B, apparatus 20 further comprises one or more robotic arms 24 mounted to chassis 22 and configured to perform robotic surgery, such as robotic ophthalmic (e.g., cataract) surgery, typically under the control of an operator. Alternatively or additionally, chassis 22 may be coupled to any other components configured to perform any other functionality.

Apparatus 20 further comprises multiple wheel units 26. For example, each of FIGS. 1A-1B shows an embodiment in which the apparatus comprises four wheel units 26, three of which are visible in the figure. As further described below with reference to the subsequent figures, wheel units 26 comprise respective wheels 28 and are configured to alternate wheels 28 between a lower position, in which the wheels support chassis 22, and an upper position. Apparatus 20 further comprises multiple legs 30 (or a single leg of sufficient size) connected to the chassis and configured to support the chassis while the wheels are in the upper position.

Various components of apparatus 20, such as a platform for supporting robotic arms 24, may limit the maximum allowable height of wheel units 26. Nonetheless, advantageously, wheel units 26 are configured to occupy relatively little vertical space, as further described below with reference to FIGS. 2A-2B.

Typically, apparatus 20 further comprises a power supply 21 configured to supply power to wheel units 26 and to circuitry configured to facilitate operation of the wheel units. Such circuitry typically includes a controller 23 and a wireless and/or wired communication interface 25. Controller 23 is configured to control wheel units 26, e.g., so as to raise or lower wheels 28, in response to signals received, via communication interface 25, from one or more sensors (examples of which are described below with reference to FIGS. 2A-2B), and/or in response to instructions received via communication interface 25. The instructions may be entered by the operator or another user, e.g., using a dedicated console, a smartphone application, or any other suitable device configured to exchange communication with the controller.

Reference is now made to FIG. 2A, which is a schematic illustration of a wheel unit 26 in which wheel 28 is in its lower position, in accordance with some embodiments of the present invention. Reference is also made to FIG. 2B, which corresponds to FIG. 2A except for some elements of wheel unit 26 being hidden from view.

In some embodiments, each wheel unit 26 comprises a rotating element 38 (FIG. 2A) coupled (e.g., via a screw) to wheel 28 from above. As indicated by a rotating indicator 39 in FIG. 2A, the rotating element is configured to rotate between a wheel-down orientation, in which the wheel is in its lower position, and a wheel-up orientation, in which the wheel is in its upper position.

In some such embodiments, wheel unit 26 comprises a pivot 36 (FIG. 2B) coupled to chassis 22. For example, chassis 22 may comprise a base 62 and a pair of pivot supports 64 mounted, at a horizontal distance from one another, to base 62, and pivot 36 may comprise a horizontal rod passing between pivot supports 64. Rotating element 38 comprises a first portion 38a, which is coupled to wheel 28, and a second portion 38b. Rotating element 38 is coupled to pivot 36 between first portion 38a and second portion 38b, such that the rotating element is configured to rotate about the pivot. In particular, in the wheel-down orientation of the rotating element, as shown in FIG. 2A, first portion 38a is at a lesser height (and second portion 38b is at a greater height), such that wheel 28 is in its lower position (and hence, supports the chassis). In contrast, in the wheel-up orientation of the rotating element, first portion 38a is at a greater height (and second portion 38b is at a lesser height), such that wheel 28 is in its upper position (and hence, does not support the chassis).

In some such embodiments, wheel unit 26 further comprises an axle 42, which is typically horizontally aligned, and an eccentric element 44, which may comprise a bearing (e.g., a metal cam bearing) for example. Eccentric element 44 is referred to as “eccentric” by virtue of the element being coupled eccentrically to axle 42. In other words, eccentric element 44 is coupled to axle 42 via an arm 46 that extends (e.g., perpendicularly) from the axle, such that the eccentric element is not aligned with the rotational axis of the axle. Thus, as the axle rotates, eccentric element 44 moves along an arced path 50 (FIG. 2B) in a vertical plane.

In such embodiments, wheel unit 26 further comprises a motor 48 coupled to chassis 22 and configured to rotate axle 42 so as to move eccentric element 44 (along arced path 50) between a supporting position and a non-supporting position. In the supporting position, as shown in FIG. 2A, the eccentric element contacts second portion 38b so as to support rotating element 38 in its wheel-down orientation. In the non-supporting position of the eccentric element, on the other hand, the eccentric element does not inhibit the rotating element from rotating to the wheel-up orientation. Motor 48 is coupled to power supply 21 (FIGS. 1A-1B) and is controlled by controller 23 (FIGS. 1A-1B).

Optionally, axle 42 may pass through one or more axle supports 40 (FIG. 2A), which are coupled to base 62 of the chassis.

Typically, in the supporting position of eccentric element 44, the eccentric element leans against chassis 22; for example, the eccentric element may lean against a stopper 52 mounted to base 62. In other words, to reach the supporting position, the eccentric element follows arced path 50 beyond the point of maximum height of the eccentric element, until the eccentric element is supported by the chassis. Thus, advantageously, much of the vertical force that would be applied to axle 42 if the eccentric element were not leaning against the chassis, is instead applied to the chassis.

Typically, the maximum height of wheel unit 26—e.g., the greater of (i) the height of first portion 38a in the wheel-up orientation, and (ii) the height of second portion 38b in the wheel-down orientation—from base 62 is less than 15 cm.

Typically, as shown in FIG. 2A, wheel unit 26 further comprises at least one tension spring 54 coupled between second portion 38b of the rotating element and chassis 22 and configured to pull down second portion 38b. Thus, advantageously, tension spring 54 may stabilize the rotating element even while the robotic surgical apparatus is wheeled over gaps in the floor.

Typically, wheel unit 26 further comprises one or more sensors. Based on the signals from the sensors, controller 23 (FIGS. 1A-1B) may ascertain the state of the wheel unit, as further described below.

In some embodiments, the sensors comprise a wheel-down sensor 56, which may be coupled, for example, to a vertical post 60 mounted to base 62. Wheel-down sensor 56 is configured to output a wheel-down signal indicating whether the rotating element is in the wheel-down orientation. For example, wheel-down sensor 56 may comprise a wired or wireless communication interface, and the wheel-down signal may be communicated to controller 23 (FIGS. 1A-1B) via this communication interface and communication interface 25 (FIGS. 1A-1B).

In general, wheel-down sensor 56 may utilize any suitable sensing technique. As a purely illustrative example, the wheel-down sensor may comprise an emitter configured to emit an electromagnetic (e.g., a visible-light) or acoustic (e.g., ultrasonic) wave, and a detector positioned opposite the emitter and configured to detect the wave. For example, in some embodiments, wheel-down sensor 56 comprises a slotted optical switch. A tab 58 may be coupled to second portion 38b of the rotating element such that, when the rotating element is in the wheel-down orientation, tab 58 is positioned between the emitter and the detector so as to block detection of the wave. Alternatively, the detector may be adjacent to the emitter, and tab 58 may reflect the wave to the detector. Alternatively, the wheel-down sensor may comprise two electrically-isolated electrically-conductive elements, and tab 58 may be electrically conductive such that, when the rotating element is in the wheel-down orientation, the tab contacts the two elements so as to complete a circuit between the elements.

In some embodiments, the sensors alternatively or additionally comprise a supporting-position sensor 66, which may be coupled, for example, to stopper 52. Supporting-position sensor 66 is configured to output a supporting-position signal indicating whether eccentric element 44 is in its supporting position. For example, supporting-position sensor 66 may comprise a wired or wireless communication interface, and the supporting-position signal may be communicated to controller 23 (FIGS. 1A-1B) via this communication interface and communication interface 25 (FIGS. 1A-1B).

In general, supporting-position sensor 66 may utilize any suitable sensing technique. Optionally, a tab 68 may be coupled to arm 46 such that, in the supporting position of the eccentric element, tab 68 is positioned with respect to sensor 66 so as to facilitate the sensing, as described above for tab 58.

In some embodiments, the sensors alternatively or additionally comprise a wheel-up sensor 70, which may be coupled, for example, to a pivot support 64. Wheel-up sensor 70 is configured to output a wheel-up signal indicating whether the rotating element is in the wheel-up orientation. For example, wheel-up sensor 70 may comprise a wired or wireless communication interface, and the wheel-up signal may be communicated to controller 23 (FIGS. 1A-1B) via this communication interface and communication interface 25 (FIGS. 1A-1B).

In general, wheel-up sensor 70 may utilize any suitable sensing technique. Optionally, a tab 72 may be coupled to second portion 38b of the rotating element such that, in the wheel-up orientation of the rotating element, tab 72 is positioned with respect to sensor 70 so as to facilitate the sensing, as described above for tab 58.

In some embodiments, the sensors comprise an imaging sensor configured to acquire an image of wheel unit 26, and the controller is configured to process the image so as to ascertain the state of the wheel unit.

Reference is now made to FIG. 3A, which is a schematic illustration of a portion of wheel unit 26, in accordance with some embodiments of the present invention.

In some cases, it may be necessary to manually lower wheel 28 such that the wheel supports chassis 22. Hence, typically, each wheel unit 26 further comprises a lever 74 coupled to chassis 22. As indicated by a rotation indicator 76, lever 74 is configured for rotation, by a user, so as to rotate the rotating element from the wheel-up orientation, which is shown in FIG. 3A.

Typically, by operating lever 74, the rotating element is rotated to a “second wheel-down orientation,” which is different from the “first wheel-down orientation” attained by the rotating element when the rotating element is rotated by the controller (e.g., using eccentric element 44). In particular, second portion 38b of the rotating element is higher (e.g., 1-10 mm higher) in the first wheel-down orientation than in the second wheel-down orientation. As further described below, this difference facilitates returning the lever to its default orientation (shown in FIG. 3A), in which the lever does not support the rotating element.

Typically, lever 74 is coupled to chassis 22 by virtue of fitting over a pivot 80, which may be coupled, for example, to the outer surface of a pivot support 64. The user grips a first end 82a of the lever or, optionally, a lever extension removably coupled to (e.g., fit over) first end 82a. As the user rotates the lever about pivot 80, the second end 82b of the lever, which is opposite the first end, pushes second portion 38b of the rotating element upward.

Typically, wheel unit 26 further comprises a tension spring 78 coupled between lever 74 and the chassis and configured to pull the lever (in particular, second end 82b) away from the rotating element. Thus, advantageously, when the lever is not in use, the lever does not destabilize the rotating element. Nonetheless, the tension force in tension spring 78 is sufficiently small such that the user may easily overcome the tension force when operating lever 74.

Reference is now also made to FIG. 3B, which corresponds to FIG. 3A except for lever 74 being hidden from view.

Typically, wheel unit 26 further comprises a locking element 84 coupled to lever 74 and configured to lock against chassis 22 such that the lever supports the rotating element following the rotation of the lever by the user. For example, chassis 22 may comprise a protrusion 86 that may protrude, for example, from pivot support 64, and locking element 84 may lock against the chassis by fitting around protrusion 86. In other words, the protrusion may fit into a portion 88 of the locking element, such that the protrusion inhibits the locking element—and hence, the lever, which is coupled to the locking element—from rotating backward (i.e., in the direction opposite to that indicated by rotation indicator 76).

Typically, wheel unit 26 further comprises a release handle 90 coupled to locking element 84 and configured for operation, by the user, so as to release the locking element from the chassis. For example, the locking element may be pivotably coupled to the lever, e.g., by virtue of a pivot, which protrudes from the lever, passing through a hole 92 in the locking element. The user may thus rotate the locking element with respect to the lever using release handle 90, thereby releasing the locking element from protrusion 86. Spring 78 may then pull second end 82b of the lever down, such that the lever rotates backward. Alternatively or additionally, using the release handle, the user may rotate the lever backward.

Typically, wheel unit 26 further comprises a tension spring 94 coupled between the locking element and the chassis (e.g., between the locking element and pivot support 64) and configured to facilitate the locking of the locking element against the chassis by pulling the locking element. For example, after locking element 84 fits around protrusion 86, spring 94 may pull the locking element toward the protrusion, such that the locking element remains fit around the protrusion. Nonetheless, the tension force in tension spring 94 is sufficiently small such that the user may easily overcome the tension force when operating release handle 90.

Typically, wheel unit 26 further comprises respective locks 96 for wheels 28, which may be operated manually so as to lock wheels 28.

As noted above, controller 23 may identify the current orientation of the rotating element based on the respective signals received, by the controller, from one or more sensors belonging to the wheel unit. For example, the controller may identify the current orientation from the three possible orientations of the rotating element described above. For example, referring again to FIGS. 2A-2B:
(i) Based on the signal from wheel-up sensor 70, the controller may ascertain that the rotating element is in the wheel-up orientation.
(ii) Based on the signal from supporting-position sensor 66, the controller may ascertain that the rotating element is in the first wheel-down orientation, indicating that the wheel was lowered automatically.
(iii) If (a) the signal from supporting-position sensor 66 does not indicate that the rotating element is in the first wheel-down orientation, but (b) the signal from wheel-up sensor 70 does not indicate that the rotating element is in the wheel-up orientation and/or the signal from wheel-down sensor 56 indicates that the rotating element is in the wheel-down orientation, the controller may ascertain that the rotating element is in the second wheel-down orientation, indicating that the wheel was lowered manually.

One case in which a manual lowering of the wheels may be required is that of a power failure. For example, at the start of a surgical procedure, the controller may raise the wheels such that the chassis is supported by legs 30 (FIGS. 1A-1B). If, however, a power failure subsequently occurs (such that the surgical apparatus becomes inoperative), it may be necessary to move the surgical apparatus away from the operating table. Hence, the wheels may be manually lowered, the apparatus may be wheeled away, and the wheels may then be locked using locks 96.

Subsequently, upon the restoration of power, the controller may ascertain that the rotating element is in the second wheel-down orientation. In response thereto, the controller may rotate the rotating element to the first wheel-down orientation, e.g., by rotating eccentric element 44 (FIGS. 2A-2B). This rotation raises the rotating element from the lever, thereby releasing some of the pressure on locking element 84 so as to enable the user to operate release handle 90 as described above. (Thus, as noted above, it is advantageous for second portion 38b to be higher in the first wheel-down orientation than in the second wheel-down orientation.) Subsequently to rotating the rotating element to the first wheel-down orientation, the controller may output an alert indicating the need to release the locking element, e.g., by operating the release handle. For example, the controller may output the alert to a computer screen.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus, comprising:
a chassis;
one or more robotic arms mounted to the chassis and configured to perform robotic surgery;
multiple wheel units comprising respective wheels and configured to alternate the wheels between a lower position, in which the wheels support the chassis, and an upper position; and
one or more legs connected to the chassis and configured to support the chassis while the wheels are in the upper position.

2. The apparatus according to claim 1, wherein a maximum height of each of the wheel units from a base of the chassis is less than 15 cm.

3. The apparatus according to claim 1, wherein each of the wheel units further comprises a rotating element coupled to the wheel of the wheel unit from above and configured to rotate between a wheel-down orientation, in which the wheel is in the lower position, and a wheel-up orientation, in which the wheel is in the upper position.

4. The apparatus according to claim 3, wherein each of the wheel units further comprises a wheel-down sensor configured to output a wheel-down signal indicating whether the rotating element is in the wheel-down orientation.

5. The apparatus according to claim 3, wherein each of the wheel units further comprises a wheel-up sensor configured to output a wheel-up signal indicating whether the rotating element is in the wheel-up orientation.

6. The apparatus according to claim 3,
wherein each of the wheel units further comprises a pivot coupled to the chassis,
wherein the rotating element comprises a first portion, which is coupled to the wheel, and a second portion, and
wherein the rotating element is coupled to the pivot between the first portion and the second portion, such that the rotating element is configured to rotate about the pivot.

7. The apparatus according to claim 6, wherein each of the wheel units further comprises:
an axle;
an eccentric element coupled eccentrically to the axle; and
a motor coupled to the chassis and configured to rotate the axle so as to move the eccentric element between a supporting position, in which the eccentric element contacts the second portion of the rotating element so as to support the rotating element in the wheel-down orientation, and a non-supporting position, in which the eccentric element does not inhibit the rotating element from rotating to the wheel-up orientation.

8. The apparatus according to claim 7, wherein each of the wheel units further comprises a supporting-position sensor configured to output a higher-position signal indicating whether the eccentric element is in the supporting position.

9. The apparatus according to claim 7, wherein, in the supporting position, the eccentric element leans against the chassis.

10. The apparatus according to claim 6, wherein each of the wheel units further comprises at least one tension spring coupled between the second portion of the rotating element and the chassis and configured to pull down the second portion of the rotating element.

11. The apparatus according to claim 6, wherein each of the wheel units further comprises a lever coupled to the chassis and configured for rotation, by a user, so as to rotate the rotating element from the wheel-up orientation.

12. The apparatus according to claim 11, wherein each of the wheel units further comprises a tension spring coupled between the lever and the chassis and configured to pull the lever away from the rotating element.

13. The apparatus according to claim 11, wherein each of the wheel units further comprises a locking element coupled to the lever and configured to lock against the chassis such that the lever supports the rotating element following the rotation of the lever by the user.

14. The apparatus according to claim 13, wherein the chassis comprises a protrusion, and wherein the locking element is configured to lock against the chassis by fitting around the protrusion.

15. The apparatus according to claim 13, wherein each of the wheel units further comprises a release handle coupled to the locking element and configured for rotation, by the user, so as to release the locking element from the chassis.

16. The apparatus according to claim 13, wherein each of the wheel units further comprises a tension spring coupled between the locking element and the chassis and configured to facilitate the locking of the locking element against the chassis by pulling the locking element.

17. The apparatus according to claim 11,
wherein the wheel-down orientation is a first wheel-down orientation, and
wherein the lever is configured for rotation so as to rotate the rotating element from the wheel-up orientation to a second wheel-down orientation in which the wheel supports the chassis,
the second portion of the rotating element being higher in the first wheel-down orientation than in the second wheel-down orientation.

18. The apparatus according to claim 17,
wherein each of the wheel units further comprises one or more sensors configured to output respective signals, and
wherein the apparatus further comprises a controller, configured to:
receive the signals from the sensors, and
based on the signals, identify a current orientation of the rotating element from a group of orientations consisting of: the wheel-up orientation, the first wheel-down orientation, and the second wheel-down orientation.

19. The apparatus according to claim 18, wherein the controller is further configured to rotate the rotating element to the first wheel-down orientation in response to ascertaining that the rotating element is in the second wheel-down orientation.

20. The apparatus according to claim 19,
wherein each of the wheel units further comprises a locking element coupled to the lever and configured to lock against the chassis such that the lever supports the rotating element in the second wheel-down orientation, and
wherein the controller is further configured to output an alert indicating a need to release the locking element, subsequently to rotating the rotating element to the first wheel-down orientation.

21. An apparatus, comprising:
a chassis;
multiple wheel units comprising respective wheels and configured to alternate the wheels between a lower position, in which the wheels support the chassis, and an upper position,
each of the wheel units further comprising a rotating element coupled to the wheel of the wheel unit from above and configured to rotate between a wheel-down orientation, in which the wheel is in the lower position, and a wheel-up orientation, in which the wheel is in the upper position; and
one or more legs connected to the chassis and configured to support the chassis while the wheels are in the upper position.
